(19) United States
(12) Patent Application Publication
(10) Pub. No.: US 2019/0083974 A1
(43) Pub. Date: Mar. 21, 2019

(54) WELL-PLATE AND FLUIDIC MANIFOLD ASSEMBLIES AND METHODS

(71) Applicant: Advanced Solutions Life Sciences, LLC, Louisville, KY (US)

(72) Inventor: Scott Douglas Cambron, Louisville, KY (US)

(73) Assignee: Advanced Solutions Life Sciences, LLC, Louisville, KY (US)

(21) Appl. No.: 16/135,299

(22) Filed: Sep. 19, 2018

Related U.S. Application Data
(60) Provisional application No. 62/560,324, filed on Sep. 19, 2017.

(51) Int. Cl. B01L 3/00; C12M 1/32

(52) U.S. Cl. CPC B01L 3/502; C12M 23/12; B01L 2200/025; B01L 2300/0829; B01L 2300/041; B01L 2200/0642

(57) ABSTRACT

A well-plate assembly includes a well-plate defining an array of wells and a fluidic manifold assembly fitted to the array of wells and configured to direct a fluid into each well of the well plate.

### CROSS-REFERENCE TO RELATED APPLICATIONS

This application claims the benefit of priority under to U.S. Provisional Application Ser. No. 62/560,324, filed Sep. 19, 2017, and entitled “Fluidic Manifold for Automated Integration of Perfusion Networks within Wellplates,” the entirety of which is incorporated by reference herein.

### TECHNICAL FIELD

The present specification generally relates to well-plate and fluidic manifold assemblies and methods, and, more specifically, well-plate and fluidic manifold assemblies and methods for perfusing structures within a well of the well-plate with a fluid.

### BACKGROUND

Well-plates are flat plates with multiple separate wells formed therein. The individual wells may be used in a variety of capacities. For example, each well may be used as a petri-dish for growing and/or printing biologic structures. Oftentimes fluid is added and or removed from the various wells of the well-plate. For example, in some cases it may be advantageous to perfuse a structure within a well of a well-plate with a fluid. Traditionally fluid may be added to a well-plate using a pipettes, syringes, or similar structures. However, because well-plates may define arrays of wells larger than 96 wells, such perfusion of individual wells may prove to be tedious. Moreover, it is difficult to standardize the pressure and/or flow rate of the fluid for the various wells or only a portion thereof.

Accordingly, a need exists for alternative well-plate and fluidic manifold assemblies for adding and/or removing fluid from a well of a well-plate.

### SUMMARY

In one embodiment, a well-plate assembly includes a well-plate defining an array of wells and a fluidic manifold assembly fitted to the array of wells and configured to direct a fluid into each well of the well-plate.

In another embodiment, a fluidic manifold assembly for a well-plate includes a manifold lid, a manifold insert, and a manifold base. The manifold insert defines a plurality of fluid flow paths. The manifold insert is positioned between the manifold lid and the manifold base. The fluidic manifold assembly is configured to be fitted to the well-plate, the well-plate having an array of wells. The plurality of fluid flow paths of the manifold insert are configured to direct fluid into each well of the well-plate.

In yet another embodiment, a method of perfusing a construct within a well of a well-plate with a fluid includes attaching a fluidic manifold assembly to the well-plate, wherein the construct is positioned within the well of the well-plate, fluidly coupling the fluidic manifold assembly to a fluid source, and priming a fluid inlet of the fluidic manifold assembly with the fluid to perfuse the construct.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a perspective view of a well-plate assembly, according to one or more embodiments shown and described herein;

FIG. 2 depicts an exploded view of the well-plate assembly of FIG. 1, according to one or more embodiments shown and described herein;

FIG. 3A depicts an underside view of a manifold plate, according to one or more embodiments shown and described herein;

FIG. 3B depicts a transparent rendering of the manifold plate of FIG. 3A, according to one or more embodiments shown and described herein;

FIG. 4 depicts a side view of the manifold plate of FIG. 3 arranged on a well-plate, according to one or more embodiments shown and described herein;

FIG. 5 depicts a perspective view of a well-plate assembly, according to one or more embodiments shown and described herein;

FIG. 6 depicts an exploded view of the well-plate assembly of FIG. 5, according to one or more embodiments shown and described herein;

FIG. 7 depicts a top view of the well-plate assembly of FIG. 5 with internal features being shown, according to one or more embodiments shown or described herein;

FIG. 8 illustrates a cross-section of the well-plate assembly of FIG. 5, according to one or more embodiments shown and described herein;

FIG. 9 illustrates a manifold assembly, according to one or more embodiments shown and described herein;

FIG. 10 illustrates a well-plate assembly incorporating the fluidic manifold assembly in an exploded view, according to one or more embodiments shown and described herein;

FIG. 11 illustrates a robotic assembly, according to one or more embodiments shown and described herein;

FIG. 12 schematically illustrates the robotic assembly, according to one or more embodiments shown and described herein;

FIG. 13A illustrates a sacrificial printed construct within a well of a well-plate, according to one or more embodiments shown and described herein;

FIG. 13B illustrates tissue construct material added to the well of FIG. 13A, according to one or more embodiments shown and described herein;

FIG. 13C illustrates a culture media solution added to the well of FIG. 13B, according to one or more embodiments shown and described herein;

FIG. 13D illustrates the sacrificial printed construct having been dissolved leaving a tissue construct with integrated channels, according to one or more embodiments shown and described herein;

FIG. 13E illustrates a manifold assembly positioned over the well-plate of FIG. 13D, according to one or more embodiments shown and described herein;

FIG. 13F illustrates fluid seeping through the tissue construct into the bottom of the well, according to one or more embodiments shown and described herein;

FIG. 13G illustrates an equilibrium position of fluid within well, according to one or more embodiments shown and described herein;

FIG. 13H illustrates the additional fluid added to the well to achieve an anticipated hydrostatic pressure, according to one or more embodiments shown and described herein;

FIG. 14 illustrates a flow chart depicting a method of perfusing a construct using a manifold assembly, according to one or more embodiments shown and described herein;

FIG. 15 illustrates a flow chart depicting a method of perfusing a construct using a manifold assembly, according to one or more embodiments shown and described herein; and

FIG. 16 illustrates a flow chart depicting a method of perfusing a construct using a manifold assembly, according to one or more embodiments shown and described herein.

### DETAILED DESCRIPTION

Embodiments described herein are directed to well-plate and fluidic manifold assemblies and methods. A well-plate assembly includes a well-plate defining an array of wells and a fluidic manifold assembly fitted to the array of wells and configured to direct a fluid into each of the wells. In embodiments, the fluidic manifold assembly distributes a fluid, for example, cell media, to printed biological structures or biological structures that are printed or grown in a lab utilizing well-plates of varying capacity. The fluidic manifold assembly may include an array of fluid inlets and outlets that are configured to interface with external hardware that may be used to perfuse desired solutions though the biological structures in the well-plates and out to containers (e.g., collection and/or disposal locations) for disposal or analytical evaluation of byproducts. Various embodiments may be employed in benchtop operations or in automated processes, as will be described in greater detail herein.

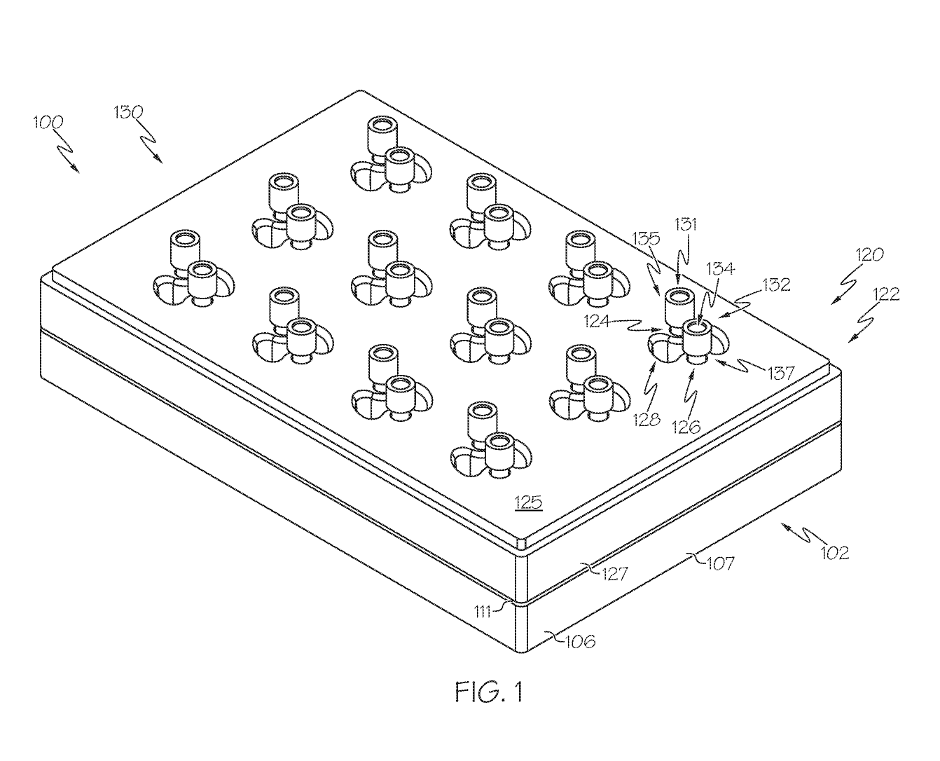

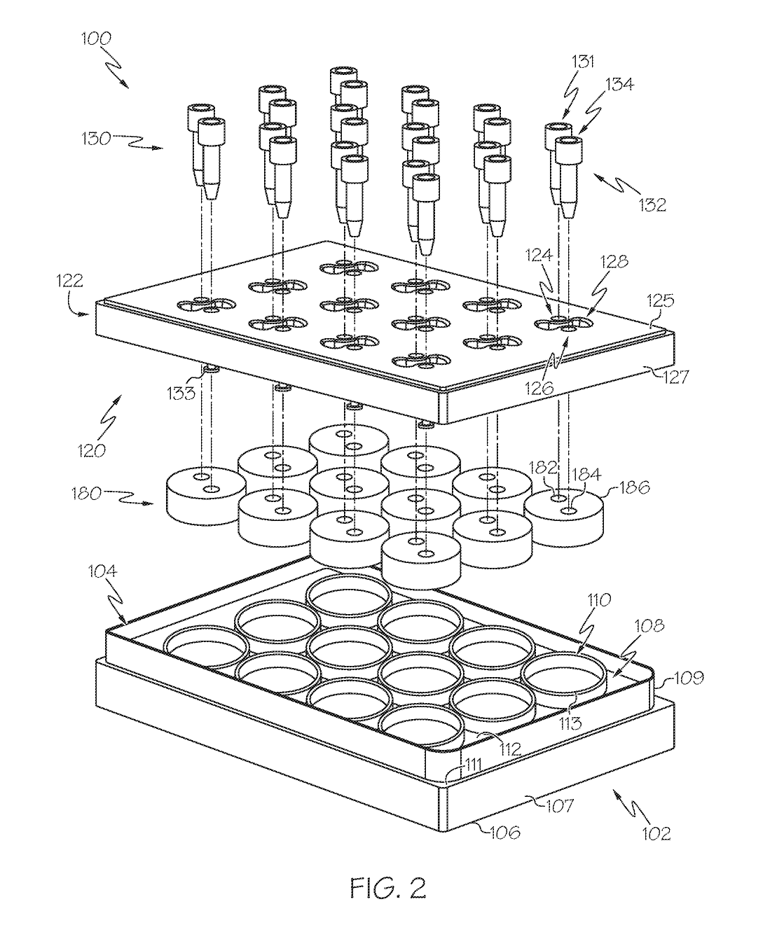

Referring now to FIG. 1, a perspective view of a well-plate assembly 100 according to one or more embodiments shown and described herein is illustrated. The well-plate assembly 100 includes a well-plate 102 and a fluidic manifold assembly 120 fitted to the well-plate 102. As will be described in greater detail herein, the fluidic manifold assembly 120 is configured to direct fluid into each of the wells 108 (illustrated in FIG. 2) of the well-plate 102. In some embodiments, the fluidic manifold assembly 120 may also be configured to direct fluid away from each of the wells 108 of the well-plate 102. In various embodiments, the various manifold assemblies described herein may be fabricated from any material suitable for providing fluid flow therethrough. For example, and not as a limitation, the various manifold assemblies may be made from a biocompatible dental grade 3-D printable resin, or the like. In other embodiments, fabrication materials may include, but are not limited to, polypropylene, polystyrene, high impact polystyrene, polyethylene, medical grade silicone rubber, resin, and combinations thereof.

It is noted that though the fluidic manifold assembly 120 is illustrated as being positioned over the array of wells 104 of the well-plate, in various embodiments, a fluidic manifold assembly may be attached beneath the well-plate. For example, a well-plate may be manufactured that has openings within the bottom of each of the wells. Accordingly, the fluidic manifold assembly may direct fluid into and/or away from the wells through the opening in the bottom of the wells.

Referring now to FIG. 2, an exploded view of the well-plate assembly 100 is depicted. From this perspective, it can be seen that the well-plate 102 defines an array of wells 104. In the present embodiment the well-plate 102 illustrates 12 individual wells 108. However, it is contemplated that well-plates may have any number of wells. For example well-plates according to the present disclosure may have 6 or more wells, 12 or more wells, 24 or more wells, 48 or more wells, 96 or more wells, etc. Each well 108 includes a well opening 110 and extends into and terminates within a body 106 of the well-plate 102. That is, the well 108 is closed at one end to retain materials therein.

A body 106 of the well-plate 102 may generally define the outer dimensions of the well-plate 102. For example, well-plates often come in standardized sizes, wherein the only change is the number of wells formed in the well-plate. That is, as the number of wells increase, the size or diameter of the wells may decrease. For example, well-plates may be about 85 mm by about 125 mm, though other sizes are contemplated and possible.

The body 106 of the well-plate 102 includes an outer wall 107 extending along an outermost perimeter of the body 106. The body 106 may include an inset wall 109 inset from the outer wall 107 such that a ledge 111 extends between the outer wall 107 and the inset wall 109. As will be described in greater detail herein, the fluidic manifold assembly 120 may extend over the inset wall 109 toward the ledge 111 to connect the fluidic manifold assembly 100 to the well-plate 102. In some embodiments, the fluidic manifold assembly 120 may extend over the inset wall 109 to be in contact with the ledge 111. In other embodiments, the fluidic manifold assembly 120 may extend over the inset wall 109 but remain spaced from the ledge 111.

In some embodiments, the body 106 of the well-plate 102 may include an upper surface 112 that the array of wells 104 extends through. In some embodiments, each well 108 may include a lip 113 that extends above the upper surface 112. In some embodiments, the inset wall 109 may also extend above the upper surface 112 of the body 106 of the well-plate 102. The distance that the inset wall 109 and the lip 113 extend above the upper surface 112 of the well-plate 102 may be substantially equal to one another or different from one another.

As noted hereinabove, the fluidic manifold assembly 120 is configured to be fitted to the array of wells 104 of the well-plate 102 and is configured to direct a fluid into each of the wells 108. In the embodiment illustrated in FIGS. 1 and 2, the fluidic manifold assembly 120 includes a fluidic manifold plate 122. The fluidic manifold plate 122 may define a plurality of fluid inlets 124 and a plurality of fluid outlets 126. In the present embodiment, the plurality of fluid inlets 124 and fluid outlets 126 are positioned in a top surface 125 of the fluidic manifold plate 122 and extends therethrough. When fitted over the array of wells 104 of the well-plate 102 each well 108 may be aligned with a fluid inlet 124 and a fluid outlet 126 such that a fluid may be added to and removed from each well 108.

The fluidic manifold plate 122 may further include a plurality of access openings. For example, between each fluid inlet 124 and fluid outlet 126 may be an access opening 128 that extends through the fluidic manifold plate 122. The access opening 128 may allow an operator to add in or remove fluid or other material manually using for example, a pipette, syringe, or similar tool. In some embodiments, there may be no access opening 128.

Extending along a perimeter of the top surface 125 of the fluidic manifold plate 122 may be a sidewall 127. As illustrated the side wall 127 may extend along an entire perimeter of the top surface 125. When assembled to the well-plate 102, the sidewall 127 may extend alongside the inset wall 109 and rest above the lip 113, as generally illustrated in FIG. 1.

The fluidic manifold assembly 120 may include a plurality of fittings 130 that may be used to discretely plumb the plurality of fluid inlets and outlets 124, 126. For example, an inlet fitting 131 may be inserted into a fluid inlet 124 and an outlet fitting 132 may be inserted into a fluid outlet 126. In some embodiments, the fluid inlet 124 and the fluid outlet 126 may be coupled to one another through a threaded engagement. The plurality of fittings 130 may be configured to fluidly couple the plurality of fluid inlets 124 to a fluid source (not shown) and the plurality of fluid outlets 126 to a desired collection location and/or disposal location. Accordingly, the plurality of fittings 130 may have a fluid passage 134 that extends therethrough to allow a fluid to flow through the plurality of fittings 130 and through the fluid outlet 126 and/or fluid inlet 124 of the fluidic manifold plate 122. For example, tubing from a fluid source (not shown) may be inserted in to the fluid passage 134 at an exposed end 135 of the inlet fitting 131 to fluidly couple the fluid inlet 124 to the fluid source. Similarly, tubing from a collection/disposal location (not shown) may be inserted in to the fluid passage 134 at an exposed end 137 of the outlet fitting 132 to fluidly couple to fluid outlet 126 to the collection/disposal location. It is contemplated that the tubing need not necessarily be attached to either the fluid source or the collection/disposal location prior to connection to the plurality of fittings 130. Instead, the tubing, as described in the below example, may be plumbed to the fluidic source and/or collection/disposal location after attachment to the plurality of fittings 130.

Shown in FIG. 2, above the array of wells 104 of the well-plate 108 are constructs 180 (e.g., printed biological tissue constructs) that are to be perfused with a fluid by the fluidic manifold assembly 120. Printed constructs and methods of fabrication are further described in U.S. patent application Ser. No. 15/202,675, filed Jul. 6, 2016, entitled “Vascularized In Vitro Perfusion Devices, Method, of Fabricating, and Applications Thereof,” hereby incorporated by reference in its entirety. Such constructs may be formed directly within a well 108 of a well-plate 102. For example, a 3-D printer (e.g., BioAssemblyBot® 3-D printing and robotics systems such as described in U.S. patent application Ser. No. 15/726,617, filed Oct. 6, 2017, entitled “System and Method for a Quick-Change Material Turret in a Robotic Fabrication and Assembly Platform,” hereby incorporated by reference in its entirety and as available from Advanced Solutions Life Sciences, LLC of Louisville, Ky.) may be used to print a sacrificial channel structure (e.g., a hydrogel such as, but not limited to, Pluronic) within each of the wells 108 of the well-plate 102. The channel structure may include a fluid channel inlet 182 and a fluid channel outlet 184 which are interconnected within the construct 180. After printing, the well 108 may be filled with biological material 186 (e.g., collagen) which is then allowed to incubate and gel. In some embodiments, the construct 180 may be allowed to incubate with the fluidic manifold assembly 120 coupled to well-plate 102 such that the fluid inlet 124 of the fluidic manifold assembly 120 may be aligned with the fluid channel inlet 182 and sealed thereto by filling the biological material above an end 133 of the fluid inlet 124. Once incubated, culture media or similar substance may then be flowed through the fluid inlet 124 and directed to the fluid channel inlet 182 to dissolve the sacrificial channel structure, leaving a network of channels within the construct 180. Various testing may then be performed on the construct 180. An example test method of utilizing the well-plate assembly 100 and fluidic manifold assembly 120 to perfuse a construct 180 is described below. It is noted that such method may similarly be applicable to other well-plate and fluidic manifold assemblies described herein.

### Example 1

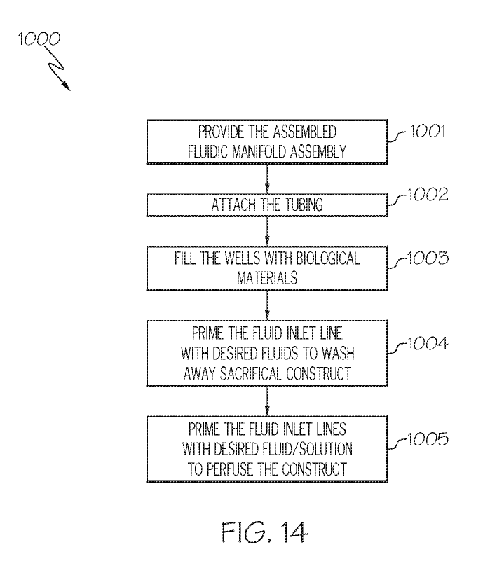

Referring to FIG. 14, a flow chart illustrating a method 1000 of utilizing of the well-plate assembly 100 of FIGS. 1 and 2 will now be described. Step 1001, provide the assembled fluidic manifold assembly 120 (e.g., the plurality of fittings 130 are threaded in respective fluid inlets and outlets 124, 126 and tubing is plumped to the plurality of fittings 130). It is noted that step 1001 may include assembling the fluidic manifold assembly. Step 1002, attach the tubing coupled to the fluid inlets 124 to the fluid source (e.g., a pump coupled to a fluid source) and attach the tubing coupled to the fluid outlets 126 to a collection and/or disposal container. Step 1003, fill the wells with biological materials (e.g., collagen) that encapsulates a sacrificial 3-D printed construct (described in greater detail herein). In some embodiments, a separate step of printing a printed construct within the well plate may be included. Step 1004, prime the fluid inlet lines (e.g., tubing, inlet fitting, fluid inlet) with a desired fluid to initially wash away the sacrificial 3-D printed construct that was previously embedded within the biological material in step 1003. Step 1005, once the sacrificial 3-D printed construct is fully washed away, prime the fluid inlet lines with desired fluid/solution to perfuse the construct with the desired fluid/solution to perform desired experimental procedures (e.g., nutritional needs/waste removal of biological constructs, mechanical conditioning of tissue, Bioreactor experiments, drug delivery to living printed/lab grown constructs, bio-analysis of how living printed/lab grown constructs metabolizes and processes various compounds within a driven fluid, etc.). It is noted that such methods may include a fewer or greater number of steps without departing from the scope of the present disclosure. Moreover, though steps are shown in a specific order, such steps may be performed in a different order without departing from the scope of the present disclosure. It is contemplated that such process may be manually achieved or automated using robotic assistance to assemble the well-plate assembly and an electronic controller (e.g., computer) to control fluid flow through the various fluid flow paths.

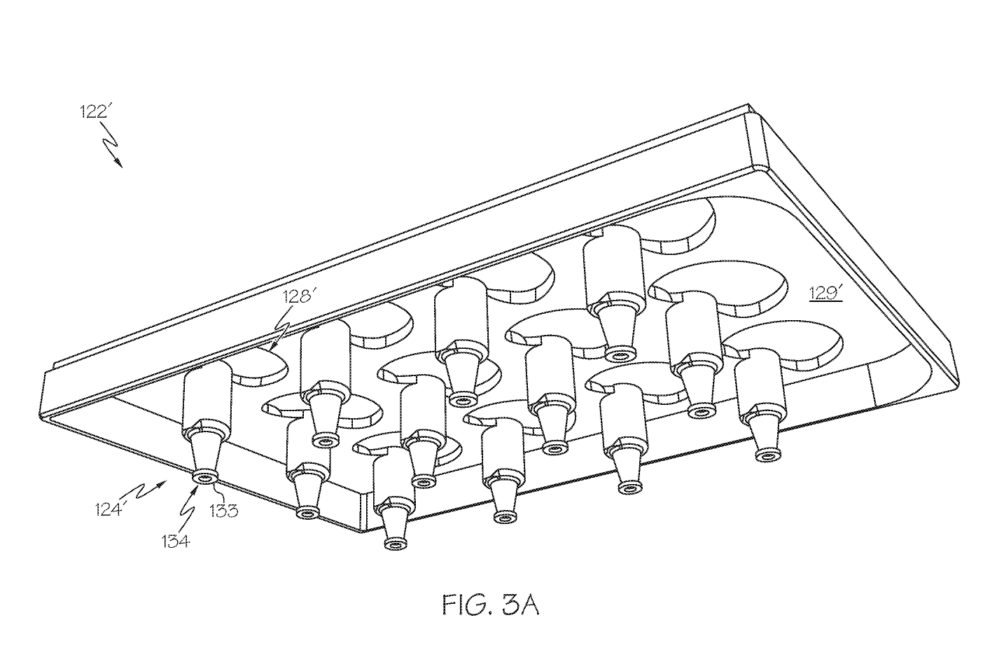

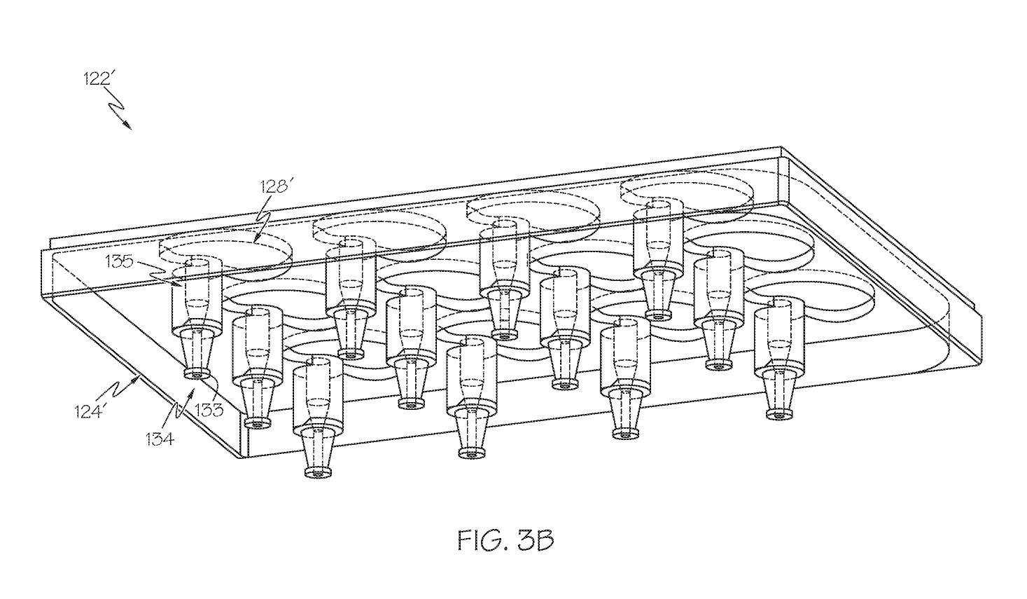

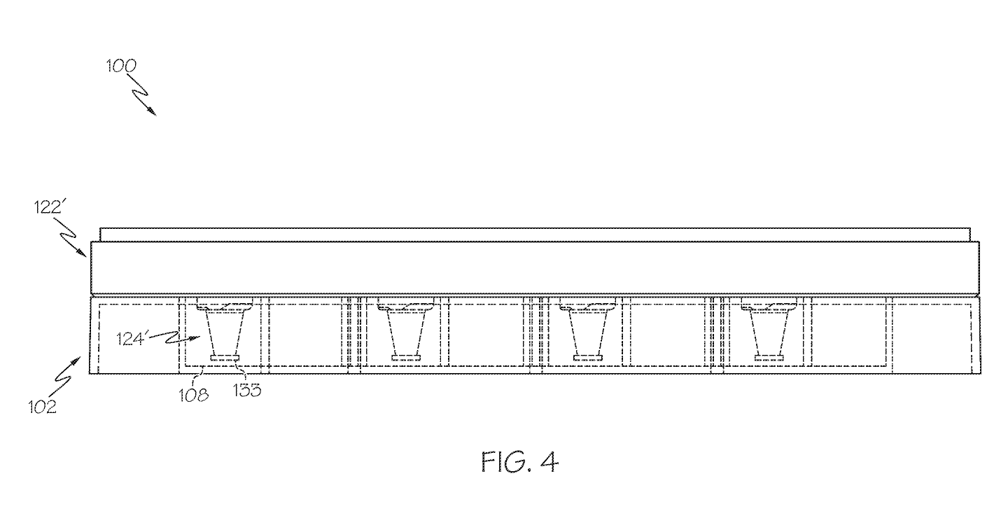

FIGS. 3A and 3B illustrate an underside surface 129′ of a fluidic manifold plate 122′. In this embodiment, the fluidic manifold plate 122′ is illustrated as only including a plurality of fluid inlets 124′. The plurality of fluid inlets 124′ extends from an underside surface 129′ of the fluidic manifold plate 122′. Accordingly, when assembled to the well-plate 102, as illustrated in FIG. 4, the plurality of fluid inlets 124′ extend into each of the wells 108 of the well-plate 102. In such embodiments, fluid removal may be achieved through the access opening 128′ using a pipette, syringe, or the like. It is noted that in embodiments with both fluid inlets and outlets as described above, the fluid outlets 126 may also extend from the underside surface 129′ of the fluidic manifold plate 122′ as illustrated for the plurality of fluid inlets 124′.

FIG. 3B is a transparent rendering of the fluidic manifold plate 122′ illustrated in FIG. 3A. From this perspective the fluid passage 134 is visible. As illustrated, the fluid passage 134 includes a threaded portion 135 for coupling the fluid passage 134 to a fitting 130 such as illustrated in FIGS. 1 and 2. It is noted that in embodiments including the plurality of fluid outlets 126, the plurality of fluid outlets 126 may include a similar structure.

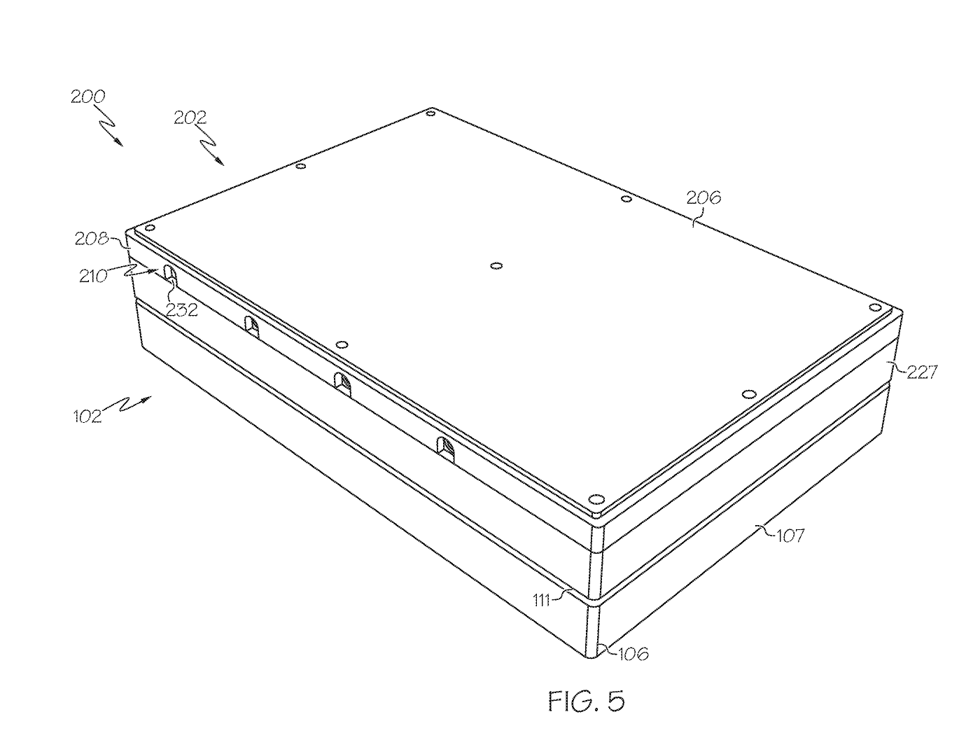

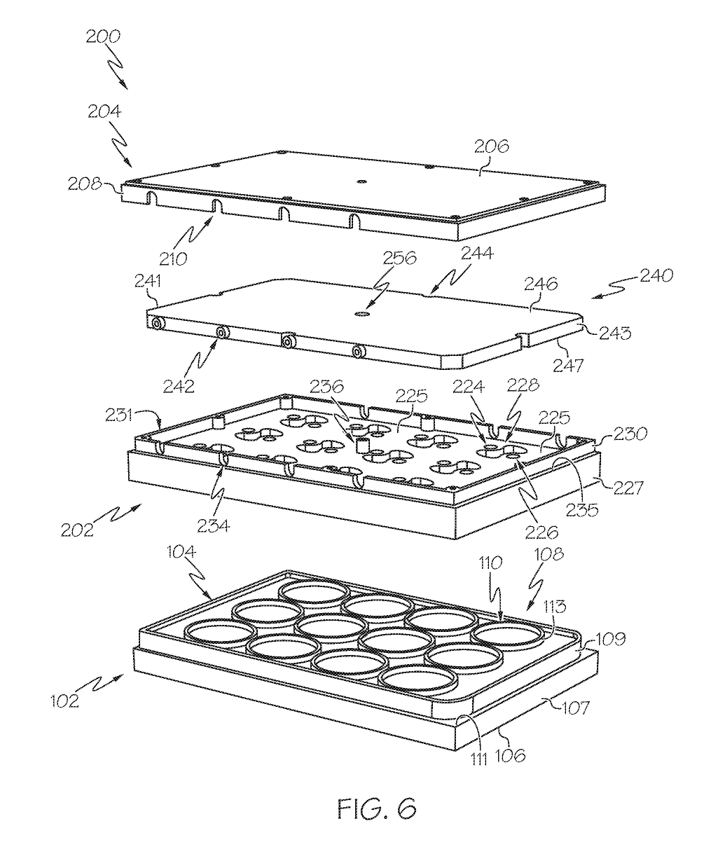

FIG. 5 illustrates another embodiment of a well-plate assembly 200. In such embodiment, the well-plate assembly 200 includes the well-plate 102, as described above in regards to FIGS. 1 and 2, and the fluidic manifold assembly 202. FIG. 6 illustrates an exploded view of the well-plate assembly 200 to illustrate additional details of the well-plate assembly 200 and the fluidic manifold assembly 202. As will be described in greater detail herein, the fluidic manifold assembly 202 may include a manifold lid 204, a manifold base 220, and a manifold insert 240. The manifold base 220 and the manifold lid 204 may form an outer clamshell housing around the manifold insert 240. Accordingly, the manifold base 220 and manifold lid 204 may encapsulate the manifold insert 240. As will be described in greater detail herein, the manifold insert 240 defines a plurality of fluid flow paths that are formed therein. The fluidic manifold assembly 202 is configured to be fitted to the well-plate 102, wherein the plurality of fluid flow paths of the manifold insert 240 are configured to direct fluid into each of the wells 108 of the well-plate 102. As will be described in more detail below, the plurality of fluid flow paths may also be configured to direct fluid away or out of each of the wells 108 of the well-plate 102.

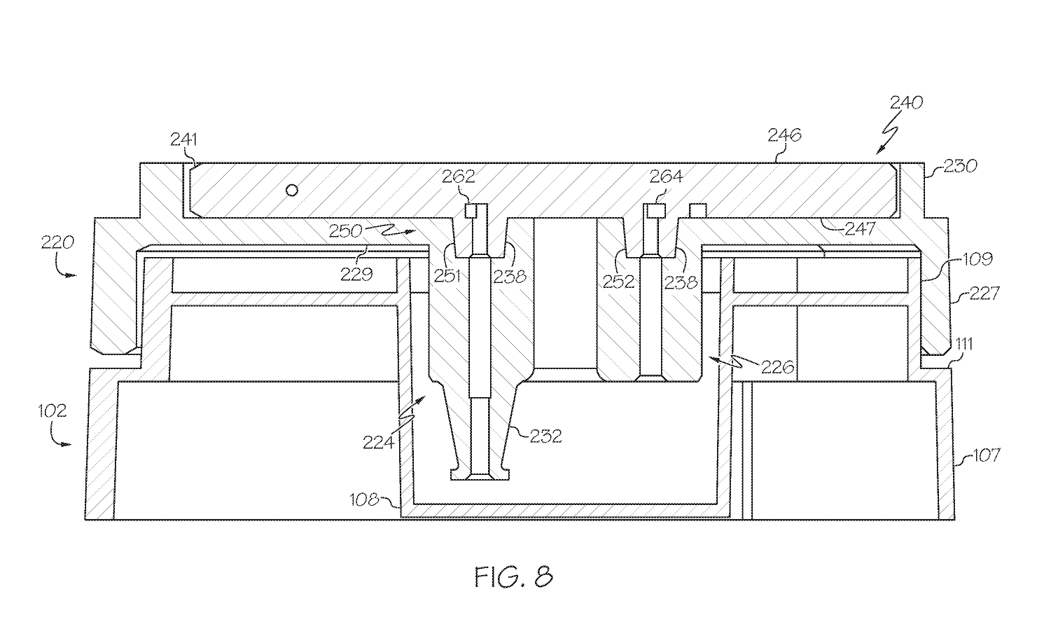

Referring collectively to FIGS. 5 and 6, the manifold base 220 is similar in structure to that described above in regards to the fluidic manifold plate 122. In particular, the manifold base 220 may define a plurality of fluid inlets 224 and a plurality of fluid outlets 226. In the present embodiment the plurality of fluid inlets and outlets 224, 226 are positioned in a top surface 225 of the manifold base 220 and extend therethrough. When fitted to the array of wells 104 of the well-plate 102 each well 108 may be aligned with a fluid inlet 224 and a fluid outlet 226 such that a fluid may be added to and removed from each well 108. FIG. 8 illustrates a cross-section of the manifold insert 240, the manifold base 220, and a single well 108 of the well-plate 102. As illustrated, the fluid inlet 224 and the fluid outlet 226 may extend from an underside surface 229 of the manifold base 220 and into the well 108 of the well-plate 102. In some embodiments, the fluid inlet 224 may extend further into the well 108 than the fluid outlet 226 or vice-a-versa. In some embodiments, the fluid inlet 224 may have a nozzle shaped portion 232 at a distal end of the fluid inlet 224. In some embodiments, the fluid inlet 224 and the fluid outlet 226 may be substantially identical. In some embodiments, the fluid inlet 224 and the fluid outlet 226 may be substantially flush with the underside surface 229 of the manifold base 220 and not extend therefrom.

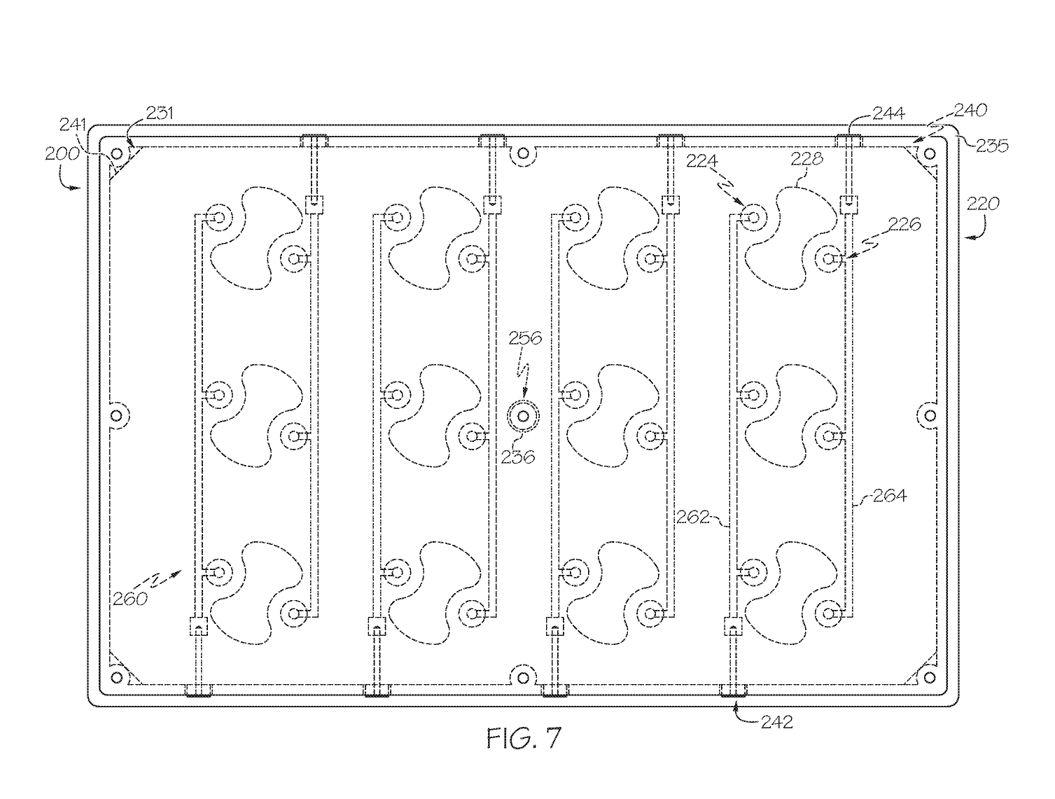

FIG. 7 transparently illustrates the fluidic manifold assembly 202 to illustrate various internal features of the fluidic manifold assembly 202. Referring collectively to FIGS. 6 and 7, between each fluid inlet 224 and fluid outlet 226 may be an access opening 228 that extends through the manifold base 220. The access opening 228 may allow an operator to add in or remove fluid or other material manually from a well 108 using for example, a pipette, syringe, or similar tool. In some embodiments, there may be no access opening 228.

Referring again to FIGS. 5 and 6, extending along a perimeter of the top surface 225 of the manifold base 220 may be a sidewall 227. As illustrated the sidewall 227 may extend along an entire perimeter of the top surface 225. When assembled to the well-plate 102, the sidewall 227 may extend alongside the inset wall 109 of the well-plate 102 toward the ledge 111, as generally illustrated in FIG. 1. It is contemplated that the sidewall 227 may rest directly atop the ledge 111 or be vertically spaced therefrom (e.g., see FIG. 8).

Still referring to FIG. 6, extending from the top surface 225 of the manifold base 220 may be a stepped-in wall 230 that is offset inward from the sidewall 227 so as to form a ledge 235 around the manifold base 220. The stepped-in wall 230 may form a dock 231 into which the manifold insert 240 may be positioned. Formed within the stepped-in wall 230 may be first plumbing openings 234 that, as will be described in greater detail herein, provide plumbing access to the manifold insert 240 to fluidly couple the manifold insert 240 to a fluid source and/or to a fluid collection/disposal location. The first plumbing openings 234 may be positioned anywhere within the stepped-in wall 230 so as to align with fluid inlet ports 242 and fluid outlet ports 244 of the manifold insert 240. It is noted that directional terms such as, “top,” “bottom,” “underside,” “upper,” and “lower” are non-limiting terms that do not limit the directional orientation of a particular piece. For example, manifold base 220 may be flipped over such the top surface 225 is spatially below underside surface 229 illustrated in FIG. 8, without departing from the scope of the present disclosure.

Extending from the top surface 225 of the manifold base 220 may be one or more alignment projections 236. The one or more alignment projections 236 may be configured to be positioned within one or more alignment recesses 256 formed within the manifold insert 240 to align the manifold insert 240 with the manifold base 220. When the fluidic manifold assembly 202 is assembled, as illustrated in FIG. 5, fasteners, pins, or the like may be passed through the manifold lid 204 and into the alignment projections 236 of the manifold base 220 to fixedly couple to manifold lid 204, the manifold insert 240, and the manifold base 220 to one another.

The manifold lid 204 forms the top enclosure of the fluidic manifold assembly 202. The manifold lid 204 includes a top wall 206 and a perimeter wall 208 extending from the top wall 206 around a perimeter of the top wall 206. When assembled to the manifold base 220 the perimeter wall 208 may extend alongside the stepped-in wall 230 of the manifold base 220 toward the ledge 235. In embodiments, the perimeter wall 208 may directly engage the ledge 235 or be spaced therefrom. Formed within the perimeter wall 208 may be second plumbing openings 210 configured to align with the first plumbing openings 234 of the manifold insert 240, so as to provide plumbing access to the manifold insert 240 to fluidly couple the manifold insert 240 to a fluid source and/or to a fluid collection/disposal location.

Referring collectively to FIGS. 6 and 7, and as noted herein, the manifold insert 240 may be configured to fit within the dock 231 defined by the stepped-in wall 230. Referring specifically to FIG. 7, the manifold insert 240 defines a plurality of fluid flow paths 260. The plurality of fluid flow paths 260 may include an inlet fluid flow path 262 and an outlet fluid flow path 264. In some embodiments, and as illustrated in the figures, the manifold insert 240 may include a plurality of inlet fluid flow paths 262 and a plurality of outlet fluid flow paths 264.

For example, in the illustrated embodiment, each row of fluid inlets 224 of the manifold base 220 includes a common inlet fluid flow path 262 and each row of fluid outlets 226 includes a common outlet fluid flow path 264. That is, each inlet fluid flow path 262 may be directed to a plurality of fluid inlets 224 of the manifold base 220 and each outlet fluid flow path 264 may be directed to a plurality of fluid outlets 226 of the manifold base 220. The embodiment illustrates four inlet fluid flow paths and four outlet fluid flow paths, but a greater or fewer number is contemplated and possible depending on the number of wells in the well-plate 102 to be included in the well-plate assembly 100. In some embodiments, it is contemplated that there may be no outlet fluid flow path.

In some embodiments, fluid inlet and fluid outlet ports 242, 244 of the manifold insert 240 may not be formed along the side wall 243 of the manifold insert 240 as illustrated in the figures, but within an upper surface 246 of the manifold insert 240. In such embodiments, the manifold base 220 and the manifold lid 204 may not include first and second plumbing openings 234, 210. Instead, plumbing openings may be provided through the top wall 206 of the manifold lid 204 or there may be no manifold lid 204 and the inlet and outlet ports 244 of the manifold insert 240 may be directly accessible.

Referring collectively to FIGS. 6 and 7, the manifold insert 240 may be configured to fit within the dock 231 defined by the stepped-in wall 230. The manifold insert 240 includes a body 241 through which the plurality of fluid flow paths 260 extend. The body 241 may define an upper surface 246, a lower surface 247, and a side wall 243 extending between the upper surface 246 and the lower surface 247. It is noted that the though terms upper and lower are used, such terms are only used in reference to the arrangement illustrated in figures are not intended to limit any orientation of the manifold insert 240.

Referring specifically to FIG. 7, the manifold insert 240 defines a plurality of fluid flow paths 260. The plurality of fluid flow paths 260 may include an inlet fluid flow path 262 and an outlet fluid flow path 264. In the illustrated embodiment, each row of fluid inlets in the manifold base 220 includes a common inlet fluid flow path and each row of fluid outlets includes a common outlet fluid flow path. That is, each inlet fluid flow path 262 may be directed to a plurality of fluid inlets 224 of the manifold base 220 and each outlet fluid flow path 264 may be directed to a plurality of fluid outlets 226 of the manifold base 220. The embodiment illustrates four inlet fluid flow paths 262 and four outlet fluid flow paths 264, but a greater or fewer number is contemplated and possible depending on the number of wells in the well-plate to be included in the well-plate assembly. In some embodiments, it is contemplated that there may be no outlet fluid flow path.

Fluid may be provided to each inlet fluid flow path 262 through a dedicated inlet port 242. Similarly, fluid may be removed from the outlet fluid flow path through a dedicated fluid outlet port 244. The inlet port 242 and the outlet port 244 are illustrated as positioned within the side wall 243. Accordingly, when the fluidic manifold assembly 202 is assembly, as illustrated in FIG. 5, the inlet port 242 and the outlet port 244 may be accessible through the overlapping first and second plumbing openings 234, 210 of the manifold base 220 and the manifold lid 204 respectively. Hypotubes or the like may be used to plumb the inlet ports 242 to the fluid source and the outlet ports 244 to collection/disposal locations. In some embodiments, it is contemplated the plurality of fluid flow paths 260 may include integrated valving to stop and/or redirect flow within the manifold insert 240.

It is noted that in some embodiments, there may be multiple manifold inserts having varying fluid networks for specific desired flows. That is different manifold inserts may be swapped out for different desired flow patterns at various times. It is contemplated the manifold insert 240 may be produced from medical grade silicone rubber, for example, or similar material.

With reference again to FIG. 8, to direct fluid into each of the wells of the well-plate, manifold insert 240 may include insert projections 250 that are configured to be mated within insert apertures 238 formed within the manifold base 220. The insert projections 250 are configured to fluidly couple the plurality of fluid flow paths with the fluid inlets and outlets 224, 226 of the manifold base 220. For example, corresponding to each well 108, the manifold insert 240 may include an inlet insert projection 251 and an outlet insert projection 252. The inlet insert projection 251 is inserted into the insert aperture 238 corresponding with the fluid inlet 224 of the manifold base 220 and the outlet insert projection 252 is inserted into the insert aperture 238 corresponding with the fluid outlet 226 of the manifold base 220. Accordingly, the inlet fluid flow path 262 of the manifold insert 240 may be fluidly coupled to the fluid inlet 224 of the manifold base 220, and the outlet fluidic flow path 264 of the manifold insert 240 may be fluidly coupled to the fluid outlet 226 of the manifold base 220.

An example test method of utilizing the well-plate assembly 200 and fluidic manifold assembly 202 to perfuse a construct (e.g., construct 180 illustrated in FIG. 2) is described below. It is noted that such method may similarly be applicable to other well-plate and fluidic manifold assemblies described herein.

### Example 2

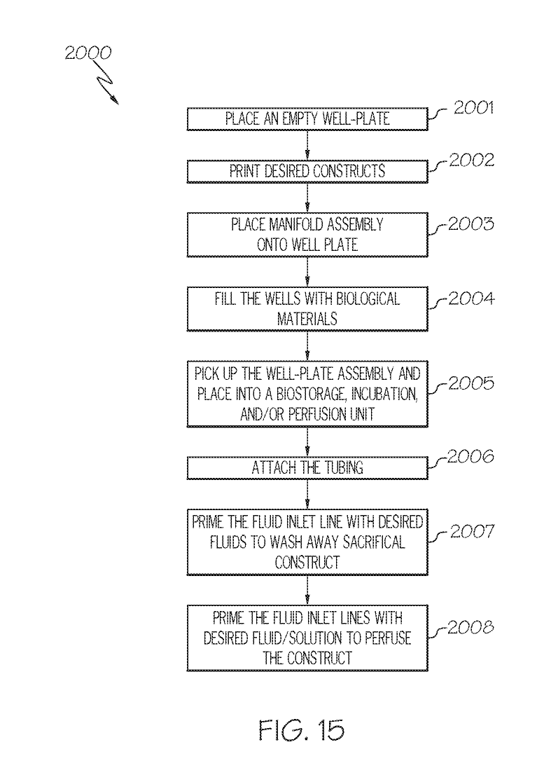

Referring to FIG. 15, a flow chart illustrating a method 2000 of utilizing of the well-plate assembly 200 will now be described. Step 2001, place an empty well-plate 102 (e.g., with robotic pick and place tool) at a desired position relative to a 3-D bioprinter. Step 2002, print desired constructs with the wells of the well-plate. Step 2003, place the manifold assembly onto well-plate (with e.g., a robotic pick and place tool such as illustrated in FIG. 11). Step 2004, fill the each well with desired biological materials (e.g., collagen) that encapsulates a printed structure (described in greater detail herein). Step 2005, pick up the entire well-plate assembly (e.g., with the robotic pick and place tool) and place into a biostorage, incubation, and/or perfusion unit. Step 2006, programmatically (e.g., with a robotic tool) connect the fluid inlets and outlets to their respective fluid sources and/or fluid collection/disposal locations with tubing (e.g., hypotubing). Step 2007, prime the fluid inlet lines (e.g., tubing, inlet fitting, fluid inlet) with a desired fluid to initially wash away the sacrificial 3-D printed construct that was previously embedded within the biological material in step 2004. Step 2008, once the sacrificial 3-D printed constructed is fully washed away, prime the fluid inlet lines with desired fluid/solution to perfuse the construct with the desired fluid/solution for desired experimental procedures (e.g., nutritional needs/waste removal of biological constructs, mechanical condition of tissue, Bioreactor, Drug delivery to living printed/lab grown constructs, bio-analysis of how living printed/lab grown constructs metabolizes and processes various compounds within a driven fluid, etc.). It is noted that such methods may include a fewer or greater number of steps without departing from the scope of the present disclosure. Moreover, though steps are shown in a specific order, such steps may be performed in a different order without departing from the scope of the present disclosure. As described above, it is contemplated that such process may be manually achieved or automated using robotic assistance to assemble the well-plate assembly and an electronic controller (e.g., computer) may be used to control fluid flow through the various fluid flow paths.

It is noted that while the above method 2000 refers to sacrificial constructs, in some embodiments, constructs may be printed using a desired material wherein the channels are formed directly within the printed construct without the need to wash away any sacrificial material.

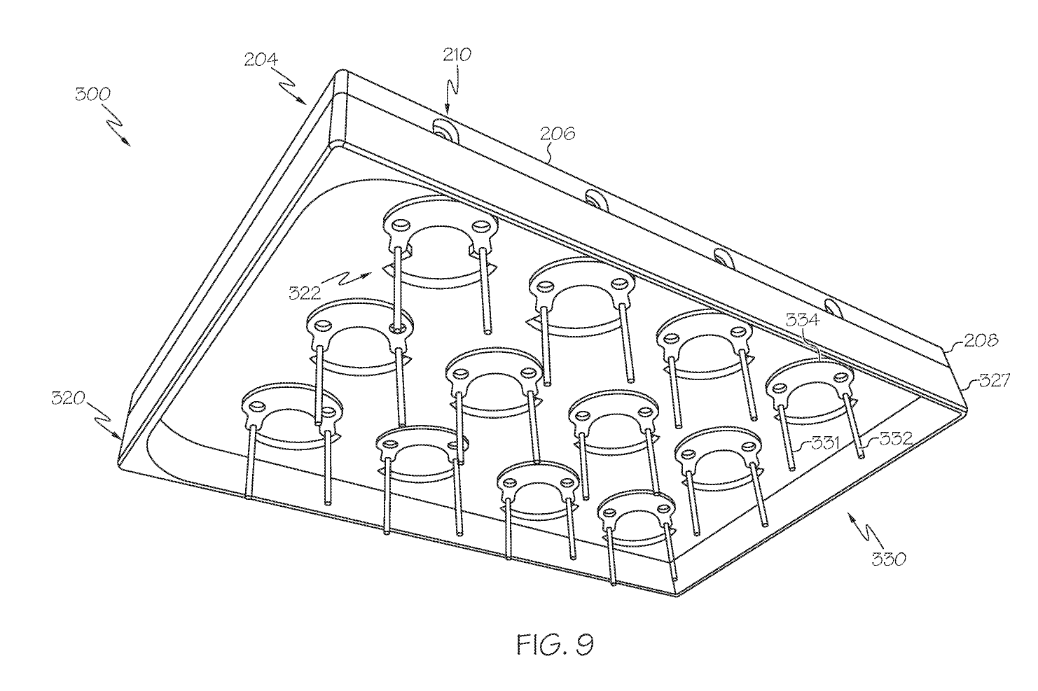

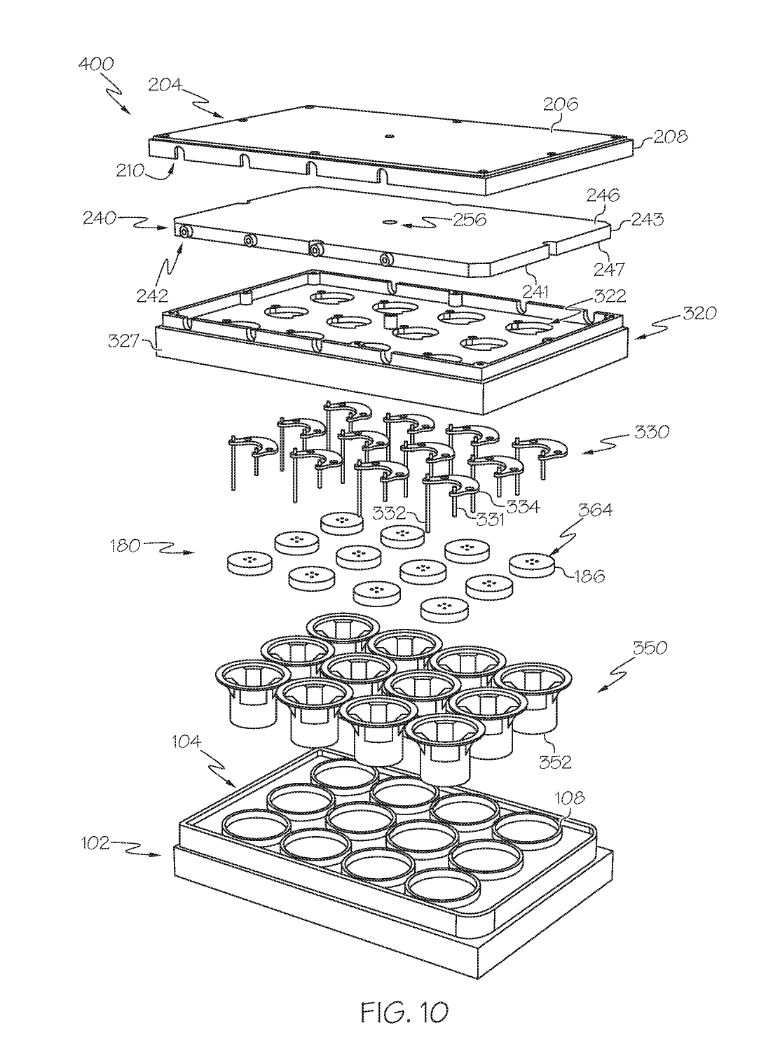

FIGS. 9 and 10 illustrate an alternative fluidic manifold assembly 300. FIG. 10 is an exploded view of the well-plate assembly 400 incorporating the fluidic manifold assembly 300. The fluidic manifold assembly 300 is similar in structure to the fluidic manifold assembly 202, described above. In particular, the fluidic manifold assembly 300 includes a manifold lid 204 and a manifold insert 240, as described above. Accordingly, regarding features of the manifold lid 204 and the manifold insert 240, such is described in greater detail above. Differences between the fluidic manifold assembly 300 and the fluidic manifold assembly will be described in greater detail below. In particular, the fluidic manifold assembly 300 further includes a manifold base 320 that is similar in structure to the manifold base 220 described above with some differences that will be described in greater detail below.

The manifold base 320 may define fluid flow apertures 322 that extend through the manifold base 320. The plurality of fluid flow apertures 322 may be configured to align with the array of wells 104 of the well-plate 102 of the well-plate assembly 200, illustrated in FIG. 10. The fluid flow apertures 322 may have any shape and are not limited to the shape illustrated in FIGS. 9 and 10. In this embodiment, the plurality of fluid flow apertures 322 replaces the above described fluid inlets and outlets 224, 226 of the manifold base 220. Instead, the fluidic manifold assembly 300 may include a plurality of hypotubes 330.

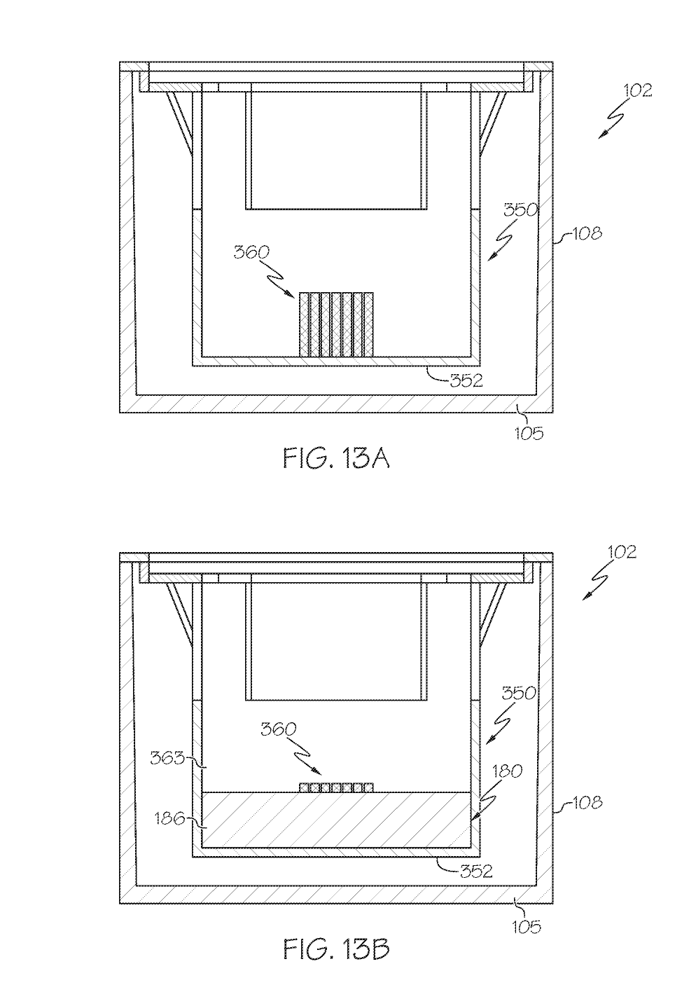

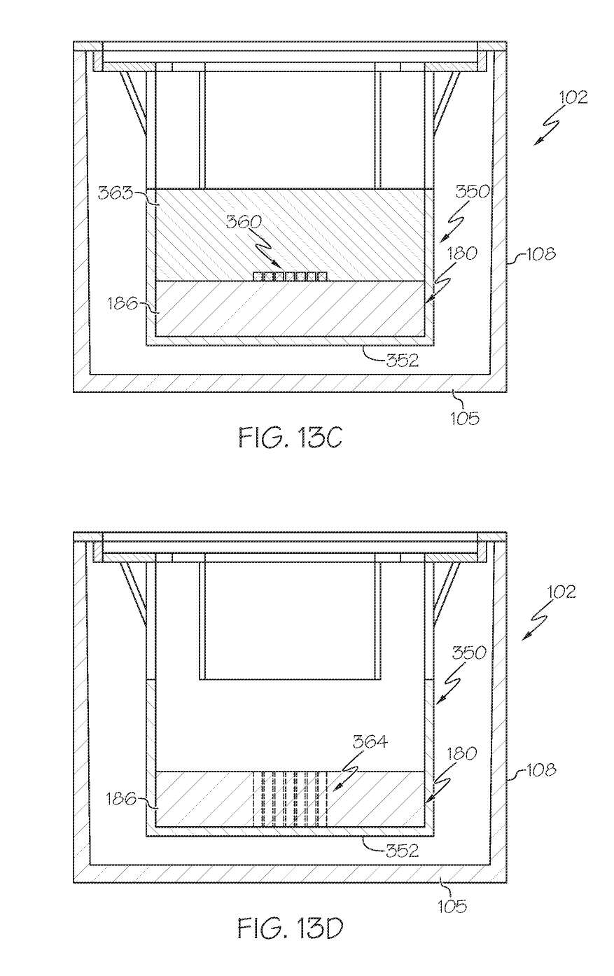

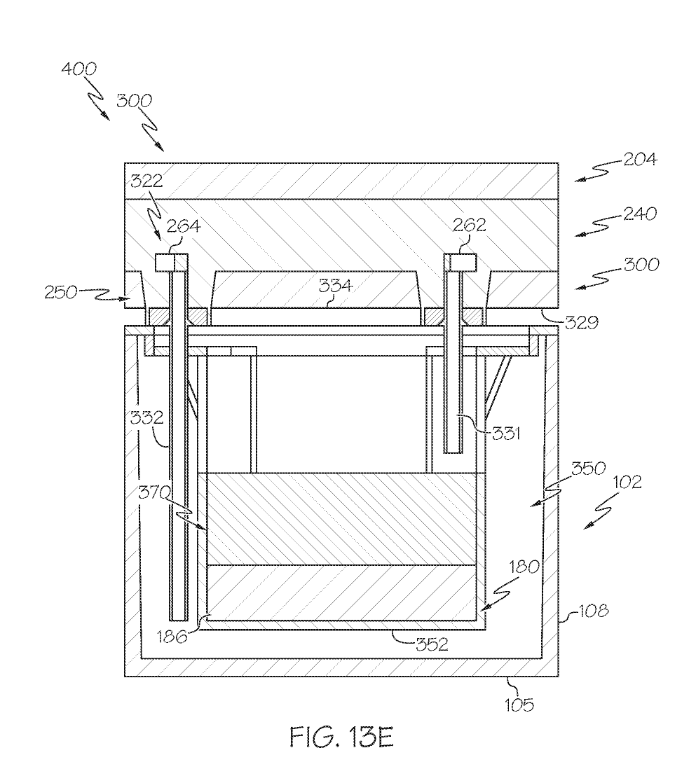

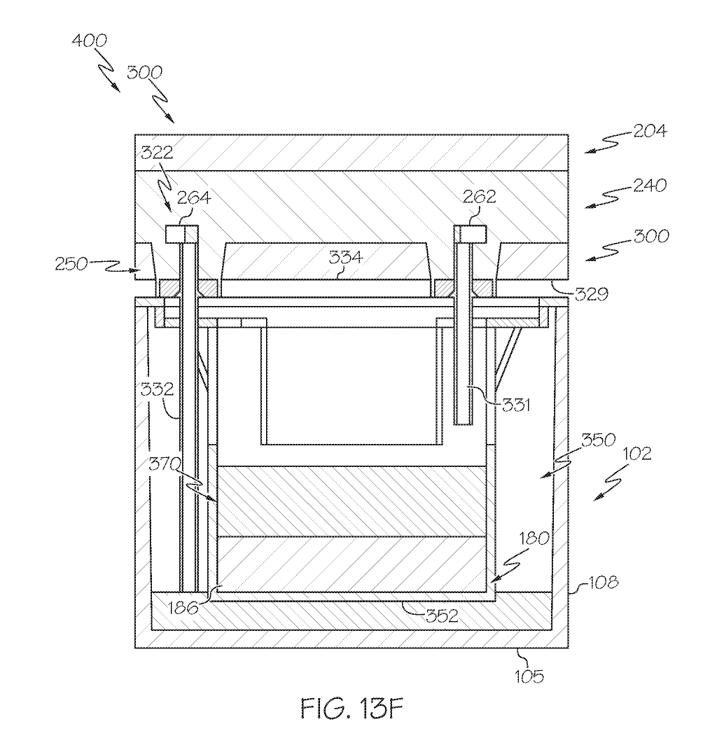

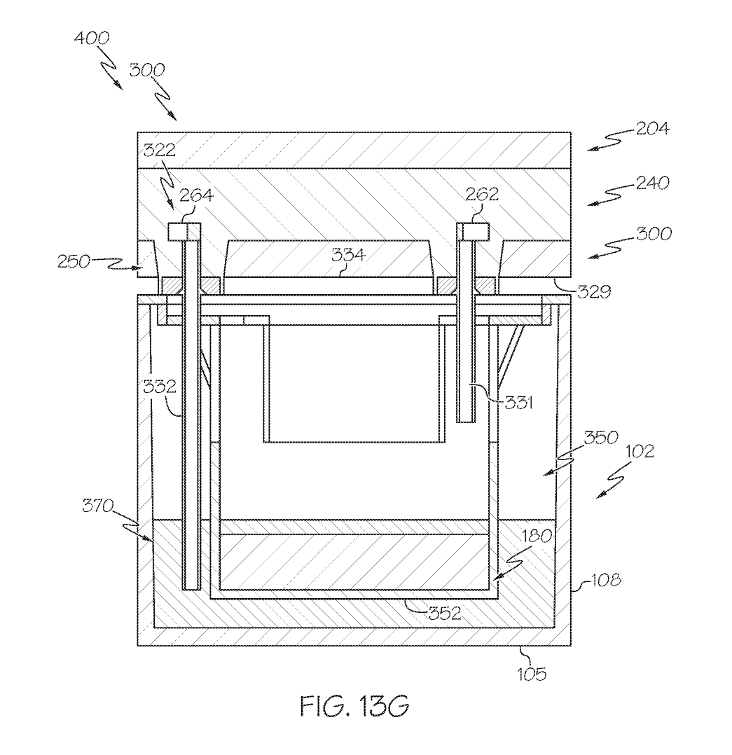

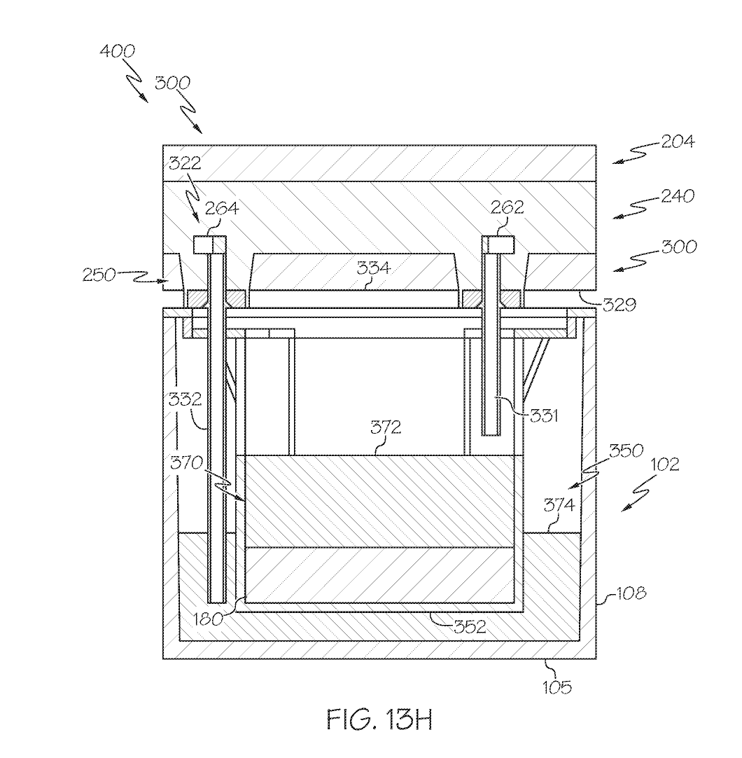

FIG. 9 illustrates the plurality of hypotubes 330 as part of the fluidic manifold assembly 300. In embodiments, the plurality of hypotubes 330 may each include an inlet hypotube 331 and an outlet hypotube 332. In some embodiments, and as illustrated in FIGS. 13A-13H, the outlet hypotube 332 may be longer than the inlet hypotube 331. The inlet hypotube 331 and the outlet hypotube 332 may be coupled to one another by a bridge member 334. In the illustrated embodiment, the bridge member is curved to complement the fluid flow aperture 322 of the manifold base 320. The use of hypotubes allow the fluidic manifold assembly 300 to have a more modular configuration so that the positions of the inlet/outlets can be easily changed and minimizes the impact (e.g., need for changes) to other portions of the fluidic manifold assembly 300 (e.g., the manifold base 320 and the manifold lid 204). That is, when a manifold insert is switched for a manifold insert having a different structure of fluid flow paths, instead of needing a different manifold base, easily swappable hypotubes may to provide the needed fluid inlets and/or outlets for a particular experimental set up.

Referring to FIG. 13E a cross-section of a single well of an assembled well-plate assembly 400 is generally depicted. In such embodiment, the insert projections of the manifold insert extend through the fluid flow aperture 322 of the manifold base 320. The inlet hypotube 331 and outlet hypotube 332 may extend into the insert projections 250 to fluidly couple the inlet and outlet fluidic flow paths 262, 264 of the manifold insert 240 to the inlet and outlet hypotubes 331, 332, respectively. When assembled, the bridge member 334 may interface directly with the underside surface 329 of the manifold base 320.

Referring again to FIG. 10, the present well-plate assembly 400 further differs from previous well-plate assemblies (e.g., well-plate assembly 200) in that the well-plate assembly 400 may further include transwells 350 that are insertable into each of the wells 108 of the array of wells 104 of the well-plate 102. Referring briefly to FIG. 13E, a cross-section of a single well 108 of the assembled well-plate assembly 400 is generally depicted. In the illustrated embodiment, the transwell 350 is positioned within the well 108 and is suspended above a base 105 of the well 108 such that there is a space between a bottom surface of the transwell 350 and the base 105 of the well 108. The bottom surface of the transwell 350 may be a transwell membrane 352 that allows fluid to pass therethrough at a predetermined rate. As will be described herein constructs 180 with integrated flow paths 188, such as described above, may be formed one the transwell membrane 352 of the transwell 350 instead of on the base 105 of the well 108 as described in the examples above.

Referring now to FIG. 11, the well-plate assembly 200/400 is illustrated with a robotic pick and place tool 500. The robotic pick and place tool 500 may be configured to interact with the well-plate assembly 200/400 and/or the fluidic manifold assembly 202/300 to transport the well-plate assembly 200/400 and or to remove/attach the fluidic manifold assembly 202/300 to the well-plate 102. For example, the manifold base 220/320 may include a grasping feature 510 such as a slot or handling recess 512. The robotic pick and place tool 500 may include grippers 502 that move relative to one another as shown to grab/and or release the grasping feature 510 of the manifold base 227/327.

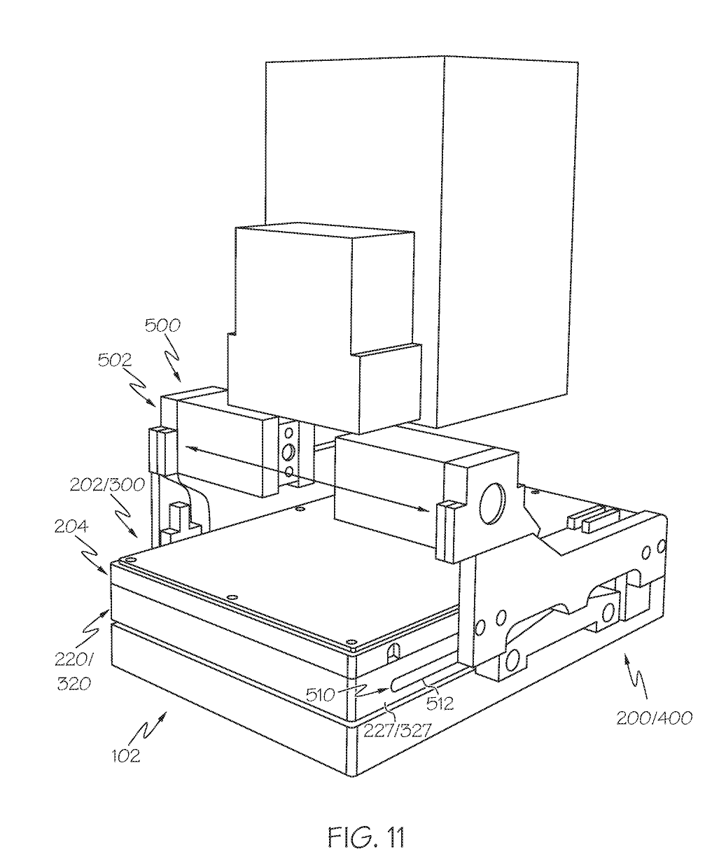

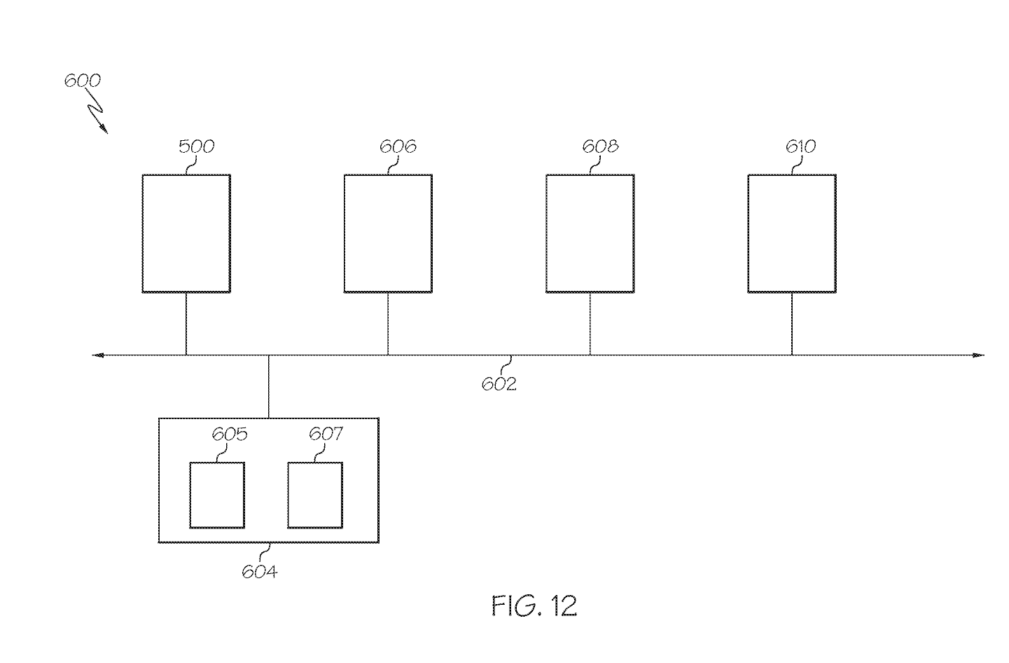

FIG. 12 schematically illustrates a system 600 for perfusing a construct within the well-plate assembly according to the various embodiments described herein. In particular, the system 600 includes a communication path 602, an electronic controller 604, the robotic pick and place tool 500, a 3-D printer 606 for printing constructs as discussed above, a fluid source 608, and one or more flow sensors 610.

The electronic controller 604 may include a processor 605 and a memory 607. The processor 605 may include any device capable of executing machine-readable instructions stored on a non-transitory computer readable medium. Accordingly, the processor 605 may include a controller, an integrated circuit, a microchip, a computer, and/or any other computing device. The memory 607 is communicatively coupled to the processor 605 over the communication path 602. The memory 607 may be configured as volatile and/or nonvolatile memory and, as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the system 600 and/or external to the system 600. The memory 600 may be configured to store one or more pieces of logic to control the various components of the system 600. The embodiments described herein may utilize a distributed computing arrangement to perform any portion of the logic described herein. Accordingly, each processor 605 may include a controller, an integrated circuit, a microchip, a computer, and/or any other computing device.

Accordingly, the electronic controller 604 may be any computing device including but not limited to a desktop computer, a laptop computer, a tablet, etc. The electronic controller 604 may be communicatively coupled to the other components of the system 600 over the communication path 602 that provides signal interconnectivity between the various components of the system 600. As used herein, the term “communicatively coupled” means that coupled components are capable of exchanging data signals with one another such as, for example, electrical signals via conductive medium, electromagnetic signals via air, optical signals via optical waveguides, and the like.

Accordingly, the communication path 602 may be formed from any medium that is capable of transmitting a signal such as, for example, conductive wires, conductive traces, optical waveguides, or the like. In some embodiments, the communication path 602 may facilitate the transmission of wireless signals, such as WiFi, Bluetooth, and the like. Moreover, the communication path 602 may be formed from a combination of mediums capable of transmitting signals. In one embodiment, the communication path 602 comprises a combination of conductive traces, conductive wires, connectors, and buses that cooperate to permit the transmission of electrical data signals to components such as processors, memories, sensors, input devices, output devices, and communication devices. Accordingly, the communication path 602 may comprise a vehicle bus, such as for example a LIN bus, a CAN bus, a VAN bus, and the like. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

The electronic controller 604 may control operations of the robotic pick and place tool 500, the 3-D printer 606, and the fluid source 608 to perform various operations. An example operation is described below. To operate each in accordance with a particular set of logic or program. For example, the electronic controller 604 may control the robotic pick and place too 500 to move the well-plate and/or fluidic manifold assembly described herein. Furthermore, the electronic controller 604 may control the 3-D printer 606 to print a desired structure (e.g., sacrificial construct for a biologic construct). The electronic controller 604 may also control the fluid source 608 to stop, reduce, and/or increase flow of fluid from the fluid source through the well-plate/fluidic manifold assembly.

As noted above, the system 600 may include one or more flow sensors 610. The one or more flow sensors 610 may include any sensor capable of outputting a signal indicative of a characteristic of the flow of fluid flowing through the well-plate and/or fluidic manifold assembly, as described above. For example, the one or more flow sensors 610 may include flow rate sensors, pressure sensors, fluid level sensors for detecting fluid height levels within the transwells 350 and/or wells of the well-plate, and the like. Based on a flow signal output by the one or more flow sensors 610 (e.g., flow rate sensors, pressure sensors, fluid height level sensors, and/or the like), the electronic controller 604 may adjust a flow of fluid traveling through the well-plate assembly by adjusting the flow of fluid from the fluid source 608.

Accordingly, the fluid source 608 may include valves, pumps, and the like that are communicatively coupled to the electronic controller 604 that allow the electronic controller 604 to control the flow of fluid through the well-plate/fluidic manifold assembly. In some embodiments, and as noted above, the fluidic manifold assembly may have integrated valves that may be controlled by the electronic controller 604 to stop or restrict the flow of fluid through the fluidic manifold assembly.

An example test method of utilizing the well-plate assembly 400 and fluidic manifold assembly 300 to perfuse a construct 180 is described below. It is noted that such method may similarly be applicable to other well-plate and fluidic manifold assemblies described herein.

### Example 3

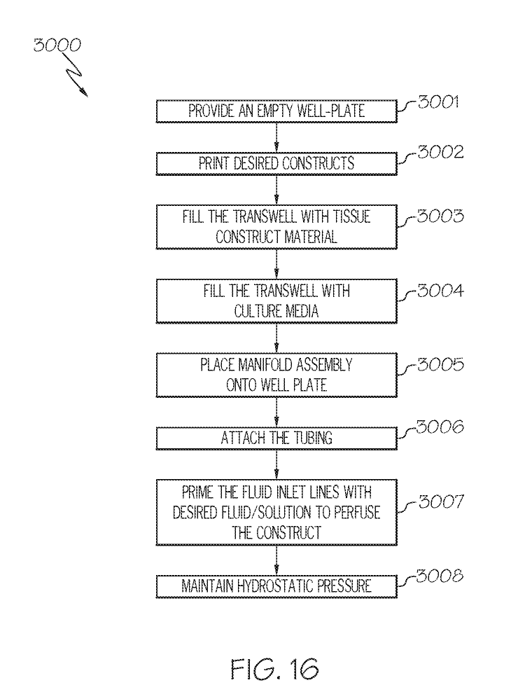

Referring to FIG. 16, a flow chart illustrating a method 3000 of utilizing of the well-plate assembly 400 in conjunction with FIGS. 13A-13H, which illustrate the method 3000 of producing and perfusing a printed construct as an example use case of the fluidic manifold assembly 300. Referring to FIGS. 16 and 13A, step 3001 includes placing the well-plate 102 with transwell 350 inserted into a well 108 onto a print stage of a 3-D printer (e.g., BioassemblyBot). At step 3002, a desired structure may be printed onto the transwell membrane 352 using for example, a soluble hydrogel such as, but not limited to Pluronic to provide a printed construct 360. Referring to FIG. 13B, at step 3003 dispense tissue construct material 186 (e.g., collagen) into the transwell 350 to encapsulated the printed construct 360. As shown the printed construct is still visible above the tissue construct material 186. The tissue construct material 186 may then be allowed to cure/gel. At step 3004 and as illustrated in FIG. 13C, the transwell 350 is filled with a culture media solution 363 configured to dissolve the sacrificial printed construct 360 leaving a tissue construct channel network 364 formed therein, as illustrated in FIG. 13D. Referring to FIG. 13E, at step 3005 the fluidic manifold assembly 300 is mounted onto the well-plate 102 such that the inlet hypotube 331 extends within the transwell 350 and the outlet hypotube 332 extends outside of the transwell 350. The fluidic manifold assembly 300 may be set in place with the robotic pick and place tool 500 (illustrated in FIG. 11) noted above or the fluidic manifold assembly 300 may be set in place manually. At step 3006, the fluidic manifold assembly 300 may be fluidly coupled to a fluid source (not shown) such the inlet fluid flow paths 262 of the fluidic manifold assembly 300 are in fluid communication with the fluid source via tubing. Similarly the fluidic manifold assembly 300 may be fluidly coupled to a collection/disposal location such that the fluid outlet fluidic flow paths 264 of the fluidic manifold assembly 300 are fluidly coupled to the collection/disposal location via tubing. Such connections are more fully described above in regards to the specific embodiments, above.

Once fluidly connected to the fluid source, at step 3007, and with reference to FIG. 13E, the transwell 350 may be filled with a fluid 370 (e.g., culture media or other desired fluid) to a desired level via the inlet hypotube 331 to achieve an anticipated hydrostatic pressure of fluid 370 on top of the tissue construct 180. The hydrostatic pressure may drive the fluid 370 at a known fluidic flow rate through the tissue construct channel network 364 and then through the transwell membrane 352 of the transwell 350 on which the tissue construct 180 is resting. With reference to FIG. 13F, the fluid 370 may be allowed to natively flow through the tissue construct channel network 364 within the tissue construct 180. If, as illustrated in FIG. 13F, inlet flow through the inlet hypotube 331 is disabled, a fluid level above the tissue construct 180 will be reduced over time and the residual culture media will collect in the bottom of the well-plate well. This gravity induced flow will continue until the fluid level above the tissue construct 180 is near the same height as the liquid level of the fluid that exits the transwell membrane 352 and collects in the well 108, as illustrated in FIG. 13G.

However, to maintain a desired hydrostatic pressure, at step 3008, the electronic controller 604, illustrated in FIG. 12, may execute logic to control the inlet and outlet flow to effectively maintain a fixed hydrostatic pressure based on a height differential between the transwell liquid level 372 and the well liquid level 374, as illustrated in FIG. 13H. For example, in some embodiments, the liquid level sensors, as noted above, may be employed to provide active feedback to the electronic controller 604 of the liquid levels in the transwell and the well-plate. Accordingly desired flow rates may be achieved through the tissue construct 180. It is noted that such methods may include a fewer or greater number of steps without departing from the scope of the present disclosure. Moreover, though steps are shown in a specific order, such steps may be performed in a different order without departing from the scope of the present disclosure.

It is noted that other possible testing may take place which does not rely on hydrostatic pressure. In other embodiments, pressure within the well-plate assembly 400 may be maintained via a pump.

Embodiments can be described with reference to the following numbered clauses, with preferred features laid out in the dependent clauses:

1. A well-plate assembly comprising: a well-plate defining an array of wells; and a fluidic manifold assembly fitted to the array of wells and configured to direct a fluid into each well of the well-plate.

2. The well-plate assembly of clause 1, wherein the fluidic manifold assembly comprises a plurality of fluid inlets and a plurality of fluid outlets.

3. The well-plate assembly of clause 2, wherein a fluid inlet of the plurality of fluid inlets and a fluid outlet of the plurality of fluid outlets are directed into each of the wells.

4. The well-plate assembly of clause 2, wherein the fluidic manifold assembly comprising a plurality of fittings coupled to the plurality of fluid inlets and the plurality of fluid outlets, and configured to fluidly couple the plurality of fluid inlets and the plurality of fluid outlets a fluid source and a collection location respectively.

5. The well-plate assembly of clause 1, wherein the fluidic manifold assembly comprises: a manifold lid; a manifold insert defining a plurality of fluid flow paths; and a manifold base, wherein the manifold insert is positioned between the manifold lid and the manifold base.

6. The well-plate assembly of clause 5, wherein: the manifold insert comprises one or more alignment recesses; and the manifold base comprises one or more alignment projections configured to be positioned within the one or more alignment recesses of the manifold insert to align the manifold insert with the manifold base.

7. The well-plate assembly of clause 5, wherein the manifold base defines a plurality of access openings.

8. The well-plate assembly of clause 1, further comprising a transwell positioned within the well of the well-plate and wherein: the fluidic manifold assembly comprises: a plurality of fluid flow paths including an inlet fluid flow path and an outlet fluid flow path; and a plurality of hypotubes, wherein a hypotube includes an inlet hypotube fluidly coupled to the inlet fluid flow path and an outlet hypotube fluidly coupled to the outlet fluid flow path, wherein the inlet hypotube directs the fluid into the transwell and the outlet hypotube removes the fluid that passes through the transwell and into the well of the well-plate.

9. A fluidic manifold assembly for a well-plate, the fluidic manifold assembly comprising: a manifold lid; a manifold insert defining a plurality of fluid flow paths; and a manifold base, wherein the manifold insert is positioned between the manifold lid and the manifold base and the fluidic manifold assembly is configured to be fitted to the well-plate having an array of wells, wherein the plurality of fluid flow paths of the manifold insert are configured to direct fluid into each well of the well-plate.

10. The fluidic manifold assembly of clause 9, wherein the plurality of fluid flow paths comprise an inlet fluid flow path and an outlet fluid flow path.

11. The fluidic manifold assembly of clause 10, wherein the manifold insert comprises: a body comprising an upper surface, a lower surface, and a side wall extending between the upper surface and the lower surface, wherein: the inlet fluid flow path extends from an inlet port at the side wall and is fluidly coupled to a fluid inlet of the manifold base; and the outlet fluid flow path extends from an outlet port at the side wall opposite the inlet port, and is fluidly coupled to a fluid outlet of the manifold base.

12. The fluidic manifold assembly of clause 9, wherein the plurality of fluid flow paths comprises a plurality of inlet fluid flow paths and a plurality of outlet fluid flow paths.

13. The fluidic manifold assembly of clause 9, wherein the manifold base defines a grasping feature, configured to be grasped by a robotic pick and place tool.

14. The fluidic manifold assembly of clause 9, wherein: the manifold insert comprises one or more alignment recesses; and the manifold base comprises one or more alignment projections configured to be positioned within the one or more alignment recesses of the manifold insert to align the manifold base with the manifold insert.

15. The fluidic manifold assembly of clause 9, further comprising a plurality of hypotubes fluidly coupled to the plurality of fluid flow paths.

16. A well-plate assembly, comprising: a well-plate defining an array of wells; a transwell positioned within a well of the well-plate; and a fluidic manifold assembly fitted to the array of wells and configured to direct fluid into each wells of the well-plate.

17. The well-plate assembly of clause 16, wherein the fluidic manifold assembly comprises: a manifold lid; a manifold insert defining a plurality of fluid flow paths; and a manifold base, wherein the manifold insert is positioned between the manifold lid and the manifold base.

18. The well-plate assembly of clause 17, wherein the fluidic manifold assembly further comprises a plurality of hypotubes fluidly coupled to the plurality of fluid flow paths.

19. The well-plate assembly of clause 18, wherein: the plurality of fluid flow paths of the fluidic manifold assembly include an inlet fluid flow path and an outlet fluid flow path; and the plurality of hypotubes each comprise an inlet hypotube fluidly coupled to the inlet fluid flow path and an outlet hypotube fluidly coupled to the outlet fluid flow path.

20. The well-plate assembly of clause 18, wherein: the plurality of fluid flow paths of the fluidic manifold assembly include an inlet fluid flow path and an outlet fluid flow path; and a hypotube of the plurality of hypotubes includes an inlet hypotube fluidly coupled to the inlet fluid flow path and an outlet hypotube fluidly coupled to the outlet fluid flow path, wherein the inlet hypotube directs the fluid into the transwell and the outlet hypotube removes the fluid that passes through the transwell and into the well of the well-plate.

21. A method of perfusing a construct within a well of a well-plate with a fluid, the method comprising: attaching a fluidic manifold assembly to the well-plate, wherein the construct is positioned within the well of the well-plate; fluidly coupling the fluidic manifold assembly to a fluid source; priming a fluid inlet of the fluidic manifold assembly with the fluid to perfuse the construct.

22. The method of clause 21, further comprising forming a construct having a channel structure formed therein within the well of the well-plate.

23. The method of clause 21, wherein the well-plate comprises a transwell positioned within the well of the well-plate, and the construct is positioned within the transwell.

24. The method of clause 21, further comprising maintaining a hydrostatic pressure within the well of the well-plate.

25. The method of clause 22, further comprising fluidly coupled the fluidic manifold assembly to a collection location.

It should now be understood that embodiments disclosed herein include various well-plate and fluidic manifold assemblies and methods. In embodiments, the fluidic manifold assembly distributes a fluid, for example, cell media, to printed biological structures or biological structures that are printed or grown in a lab utilizing well-plates of varying capacity. The fluidic manifold assembly may include an array of fluid inlets and outlets that are configured to interface with external hardware that may be used to perfuse desired solutions though the biological structures in the well-plates and out to containers for disposal or analytical evaluation of byproducts. The fluidic manifold assemblies as described herein may be used to test various constructs and provide more precise and repeatable experiments on a larger scale.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A well-plate assembly comprising:
a well-plate defining an array of wells; and
a fluidic manifold assembly fitted to the array of wells and configured to direct a fluid into each well of the well-plate.

2. The well-plate assembly of claim 1, wherein the fluidic manifold assembly comprises a plurality of fluid inlets and a plurality of fluid outlets.

3. The well-plate assembly of claim 2, wherein a fluid inlet of the plurality of fluid inlets and a fluid outlet of the plurality of fluid outlets are directed into each of the wells.

4. The well-plate assembly of claim 2, wherein the fluidic manifold assembly comprising a plurality of fittings coupled to the plurality of fluid inlets and the plurality of fluid outlets, and configured to fluidly couple the plurality of fluid inlets and the plurality of fluid outlets a fluid source and a collection location respectively.

5. The well-plate assembly of claim 1, wherein the fluidic manifold assembly comprises:
a manifold lid;
a manifold insert defining a plurality of fluid flow paths; and
a manifold base, wherein the manifold insert is positioned between the manifold lid and the manifold base.

6. The well-plate assembly of claim 5, wherein:
the manifold insert comprises one or more alignment recesses; and
the manifold base comprises one or more alignment projections configured to be positioned within the one or more alignment recesses of the manifold insert to align the manifold insert with the manifold base.

7. The well-plate assembly of claim 1, wherein the fluidic manifold assembly is configured to direct the fluid away from each of the wells.

8. The well-plate assembly of claim 1, further comprising a transwell positioned within the well of the well-plate and wherein:
the fluidic manifold assembly comprises:
a plurality of fluid flow paths including an inlet fluid flow path and an outlet fluid flow path; and
a plurality of hypotubes, wherein a hypotube includes an inlet hypotube fluidly coupled to the inlet fluid flow path and an outlet hypotube fluidly coupled to the outlet fluid flow path, wherein the inlet hypotube directs the fluid into the transwell and the outlet hypotube removes the fluid that passes through the transwell and into the well of the well-plate.

9. A fluidic manifold assembly for a well-plate, the fluidic manifold assembly comprising:
a manifold lid;
a manifold insert defining a plurality of fluid flow paths; and
a manifold base, wherein the manifold insert is positioned between the manifold lid and the manifold base and the fluidic manifold assembly is configured to be to the well-plate, the well-plate having an array of wells, wherein the plurality of fluid flow paths of the manifold insert are configured to direct fluid into each well of the well-plate.

10. The fluidic manifold assembly of claim 9, wherein the plurality of fluid flow paths comprise an inlet fluid flow path and an outlet fluid flow path.

11. The fluidic manifold assembly of claim 10, wherein the manifold insert comprises:
a body comprising an upper surface, a lower surface, and a side wall extending between the upper surface and the lower surface, wherein:
the inlet fluid flow path extends from an inlet port at the side wall and is fluidly coupled to a fluid inlet of the manifold base; and
the outlet fluid flow path extends from an outlet port at the side wall opposite the inlet port, and is fluidly coupled to a fluid outlet of the manifold base.

12. The fluidic manifold assembly of claim 9, wherein the plurality of fluid flow paths comprises a plurality of inlet fluid flow paths and a plurality of outlet fluid flow paths.

13. The fluidic manifold assembly of claim 9, wherein the manifold base defines a grasping feature, configured to be grasped by a robotic pick and place tool.

14. The fluidic manifold assembly of claim 9, wherein:
the manifold insert comprises one or more alignment recesses; and
the manifold base comprises one or more alignment projections configured to be positioned within the one or more alignment recesses of the manifold insert to align the manifold base with the manifold insert.

15. The fluidic manifold assembly of claim 9, further comprising a plurality of hypotubes fluidly coupled to the plurality of fluid flow paths.

16. A method of perfusing a construct within a well of a well-plate with a fluid, the method comprising:
attaching a fluidic manifold assembly to the well-plate, wherein the construct is positioned within the well of the well-plate;
fluidly coupling the fluidic manifold assembly to a fluid source;
priming a fluid inlet of the fluidic manifold assembly with the fluid to perfuse the construct.

17. The method of claim 16, further comprising forming a construct having a channel structure formed therein within the well of the well-plate.

18. The method of claim 16, wherein the well-plate comprises a transwell positioned within the well of the well-plate, and the construct is positioned within the transwell.

19. The method of claim 16, further comprising maintaining a hydrostatic pressure within the well of the well-plate.

20. The method of claim 16, further comprising fluidly coupled the fluidic manifold assembly to a collection location.
